(19) United States
(12) Patent Application Publication
(10) Pub. No.: US 2025/0149174 A1
(43) Pub. Date: May 8, 2025

(54) PREDICTIVE MACHINE LEARNING MODELS BASED ON DATA COLLECTED FROM A VARIETY OF SOURCES

(71) Applicant: Tibi Health Inc., San Francisco, CA (US)

(72) Inventors: Simal OZEN IRMAK, San Francisco, CA (US); Utku Irmak, San Francisco, CA (US)

(21) Appl. No.: 18/900,480

(22) Filed: Sep. 27, 2024

Related U.S. Application Data
(63) Continuation-in-part of application No. 17/516,477, filed on Nov. 1, 2021, now abandoned.
Application No. 17/516,477 is a continuation of application No. PCT/US2021/055825, filed on Oct. 20, 2021.
(60) Provisional application No. 63/104,364, filed on Oct. 22, 2020.

(51) Int. Cl. G16H 50/20; G16H 10/60; G16H 20/00

(52) U.S. Cl. CPC G16H 50/20; G16H 10/60; G16H 20/00

(57) ABSTRACT

The computer-based methods and systems presented in this disclosure provide prediction occurrence of an event for an individual on a user device of the individual. The system receives, from a plurality of remote devices, pieces of input data about the individual. The system pre-processes the pieces of input data to make them ready to be processed by respective input modules of a machine learning model running on the system. Each input module is associated with a respective marker and processes the pre-processed data for that marker. Outputs of the input modules are further processed by the model. The model provides an output indicating respective probabilities that particular events happen. The system can generate one or more alerts based on the output of the model, and can send the alerts to contacts of the individual.

### BACKGROUND

Artificial intelligence plays a significant role in data analysis and in providing predictions in many fields of technologies. Data can be obtained automatically, e.g., through sensors, or manually, e.g., through data entry by an operator. Input data can have different formats. For example, sensors can have different types, that each can produce a different data format. A sophisticated machine learning model would be able to provide outputs based on analysis of as many relevant data as possible regardless of the data types or formats.

### SUMMARY

Implementation of the present disclosure provide machine learning models that are able to obtain input data of different types, and provide outputs by analyzing those data without having to skip a particular data due to its format or type. The output of a model can be a prediction of an event happening based on the analysis of data received from a variety of sources, e.g., different types of sensors, different mobile applications, etc.

The model applies multiple layers of neural networks on the input data to identify and extract respective conditions for multiple markers, to determine respective probabilities of the particular events happening based on the respective markers, and to output respective final probabilities of the particular events happening based on the probabilities determined for the multiple markers.

In some implementations, the machine learning model includes an input layer that by itself includes several modules each running a respective neural network. Each of those input modules can analyze input data received from a variety of input resources, but for a particular marker associated with that particular input module.

The input data is pre-processed to obtain input for the input layer. The pre-processing includes extracting from a piece of input data multiple features associated with a particular input module, and transforming each extracted feature to a respective input feature that has a respective format associated with the input module. Each input module provides the probabilities of the particular events happening with respect to the marker associated with that input module.

The outputs of the input modules are inputted to hidden layers of the machine learning model. The model outputs respective probabilities of each of the particular events happening based on the probabilities received form the input modules.

Some implementations of the present disclosure include methods and computing systems capable of diagnosing and/or predicting behavioral (such as mental) disorders in an individual based on information received about the individual from different sources. The sources can be remote from the computing system, and can provide raw input data in a variety of formats. Examples of the raw input sources include sensors that monitor the individual's behavior or health, the individual's self-reporting entries, data received from members in the circle of friends and close families of the individual, healthcare providers of the individual, etc.

The presented computing system runs complex, multi-layered, multi-neural network models to provide an accurate diagnosis/prediction for the individual's behavioral conditions. The model is capable of providing respective probabilities for the multiple disorders concurrently. The model applies multiple layers of neural networks on the input data to identify and extract respective health conditions for multiple health markers, to determine respective probabilities of multiple disorders based on respective health markers, and to output respective final probabilities of the multiple disorders based on the probabilities determined for different health markers.

The present disclosure also introduces a platform that allows its users to benefit from the computing system's diagnosis and/or prediction functions. A user can identify a circle of their friends and families on the platform. Each of the individual and/or the members in the individual's circle can enter to the platform (e.g., through applications running on their respective computing devices) their observations of the individual's well-being and behavior at any time. The computing system receives that data to evaluate the individual's health status. In addition to diagnosis and/or prediction functions, the computing system can provide recommendations on what treatment or preventative actions the individual can take with respect to one or more of the behavioral disorders.

Among other advantages, the implementations can provide one or more of the following benefits.

First, the present implementations can reduce delayed, missed, or mis-diagnosis resulting from common delays in the conventional healthcare system, particularly, in the mental health field. Some of the errors that happen in diagnosing mental/behavioral disorders are rooted in incomplete information about the target individual's health and life routines. The incomplete information challenge can continue during the treatment phase as well. Health providers generally rely on a single or very few points-in-time, highly subjective assessments at individual appointments to guide the treatment course and to detect behavioral health crisis situations. This results in considerable cases of mistreatment, for example treatments with a medication that is counter-indicative of a person's true disorder. Some patients never receive the right therapeutic care and reach complete symptom resolution over decades of treatment. And many preventable crisis situations are missed, leading to more critical issues such as suicide and substance use.

The present implementations overcome these challenges that affect clinicians' judgments and recommended treatments. The techniques provide the users (e.g., target individuals) the opportunity to enter their concerns or details about their life routines and health issues to their personal devices, and receive information about the probability of suffering (currently or in the future) from certain behavioral disorders. The techniques reduce the delay, the possibility of missing a diagnosis, and the likelihood of mis-diagnosing a behavioral disorder because the information received about the target individual can be accumulated over time, can be obtained from multiple sources (e.g., friends and family of the individual, sensors attached to or monitoring certain health features of the individual, etc.), and can be analyzed in a multi-lateral format.

Second, the present techniques can review the input data for multiple disorders concurrently. This is another aspect that reduces missed diagnosis, or mis-diagnosing of a particular disorder because health providers usually focus on evaluating an individual for certain disorders while missing to consider other disorders or missing the data needed to consider other disorders.

Third, the implementations reduce subjectivity in the diagnosing or predicting behavioral disorders. Diagnosing a health disorder through conventional procedures highly depends on the healthcare provider's experience and knowledge.

The implementations also reduce mis-diagnosis and/or mis-treatments rooted in potentially misleading subjective assessment of an individual from their self-evaluation. Conventionally, health providers often rely on the subjective accounts of the individual, which can be biased and limited by the cognitive and affective ability, with limited (if any) access to the individual's health records. For example, a provider has to operate on the subjective information coming from the individual, who has been experiencing conditions affecting their emotional state as well as cognition. An individual may intentionally or unintentionally not share some information at a certain point in time or at certain appointments. Their attention, consciousness, reasoning, perception, judgment and hence experience of events or memory of facts may not be reliable. The provider, however, has to rely on the subjective accounts of the individual to recognize a constellation of symptoms that meet certain diagnostic criteria, to eliminate disorders with overlapping patterns of symptoms, and to discern primary conditions from secondary conditions based on the timeline of emergence of relevant symptoms.

The implementations reduce the effect of anyone's subjective evaluation on evaluating an individual's mental health because the implementations base their diagnosis/prediction of an individual from multiple different sources. The opinion of the individual's health provider, or the individual's self-evaluation are only a few of the many inputs the implementations receive to make a disorder diagnosis/prediction for the individual. Further, since the system can re-train itself based on its past diagnosis/predictions and the individual's health progress, the system only gets more objective as the time passes.

Fourth, some implementations in the present disclosure provide diagnosis or disorder prediction based on temporal data gathered over a period of time rather than based on point-in-time clinical impressions of the individual in individual appointments (as is common in the conventional health care system). Using temporal data also reduces mis-diagnosing an individual based on their single point-in-time evaluations.

Fifth, the presented implementations are scalable. The implementations include large-scale data analysis that is beyond the ability of any human healthcare provider. The input data about an individual can be received from multiple sources, which can add up (e.g., over time) to an amount beyond a human's capability to analyze. In some implementations, the multiple layers of neural networks are nested to make sure the effect of each piece of input data is thoroughly considered for different disorders. Also, since the input data is received from different sources, the data formats can differ from each other, which adds to the complexity of the data analysis process.

The presented implementations are also economically more affordable to the users than the conventional healthcare diagnostic and treatment procedures. The scalability of the present techniques allows using them for a larger number of patients than a number that any conventional healthcare provider could handle. Instead of paying a few visits to a health-care provider to get diagnosed, a user of the platform presented in this disclosure can use the platform over and over, which would significantly reduce the diagnosis costs. Since the user can use the platform continuously (i.e., over the course of days or months rather than a one time use), the user's health status is continuously monitored and updated with a much lower cost as compared to repeatedly visiting a healthcare provider.

Sixth, some of the implementations provide recommendations on actions to be taken to treat, reduce progression rate, or prevent development of certain behavioral disorders for an individual. In addition to the economic values that such recommendations would provide, they can offer more diverse options (e.g., physical activity, certain medication, certain procedure or surgeries, etc.) that the individual can choose from. The chance of recommending conflicting medication and treatments to the same individual would also be reduced as compared to a human provider prescribing certain medications.

Seventh, the diagnosis, predictions and/or recommendations that the implementations provide, factor in the details surrounding an individual's life, and thus, are more personalized as compared to a diagnosis or recommendation that the individual would receive at individual doctor appointments. For similar reasons, the present techniques can provide a higher rate of symptom resolution, faster recovery, and a lower risk in comorbid conditions.

Eighth, the implementations save time and energy, and provide a more comfortable communication channel for patients. Going to a clinic can cause feeling embarrassed, be internally or externally stigmatized, be logistically not plausible or time and energy consuming, which may tempt an individual to postpone taking action for their noticed behavioral disorders. Such postponement delays their diagnosis, and can cause the progress of a disorder. Further, the individual may feel shy or miss sharing certain health events with a healthcare provider at a face to face appointment.

By using the platform discussed in this disclosure, the individual can simply enter their symptoms, their state of mind, their life routines, or even their diaries into their personal device, and receive a health evaluation on the device through the platform. The implementations are particularly beneficial for individuals who need to be treated, yet have little to no time to spend for themselves. An example of such individuals is mothers of newborns who may suffer from postpartum mental disorders such as anxiety, depression, etc., yet have no awareness of their mental status or have little to no time to leave home and seek medical attention through the conventional mental healthcare system. The consequences of postpartum mental disorders can be harsh (e.g., attempts to suicide, infanticide, or harm to the child) but can be eliminated through an on-time diagnosis and treatment, which is made possible by the present implementations.

Ninth, the implementations are accessible on user's personal devices, and works for anyone regardless of their health background. The platform benefits anyone, regardless of whether they do or do not have a history of a mental disorder. Conventional healthcare system is designed for “patients”—i.e., individuals, who are already recognized as “patients” in the system and had previously sought treatments, or have had behavioral health interactions. That system does not work in “off-patient” settings where at-risk individuals could be regularly screened and triaged for prevention before they become a “patient.” Hence, prevention of the development of preventable conditions through timely detection and intervention of subsyndromal symptoms is extremely rare in the conventional system. In the present implementations, however, anyone-regardless of their background health or history of healthcare usage—can benefit from the platform and get diagnosed or evaluated for their mental health status.

The implementations are particularly beneficial in behavioral health crisis triage. There is no conventional effective and desirable triage care to address emergency situations such as suicide and psychosis. Unless “existing patients” have not presented suicidal behavior or state of psychosis during a healthcare appointment, emergency services (such as engagement of police, psychiatric holds, involuntary hospitalizations) that are known to be sub-optimal and often further traumatizing are the established pathways for crisis care. Through the present implementations, however, an individual is monitored routinely regardless of whether they show alerting signs of behavioral health crisis at an appointment. Alerts would be sent to the individual's contact(s) in case of detecting the possibility of a crisis. The alerts can include recommendations on how to approach the identified crisis to reduce potential (unintended) consequences of conventional crisis interventions (e.g., traumatizing).

Tenth, the implementations integrate the evaluations and entries received from close friends and family, into the diagnosis/prediction calculations. This is a critical piece of information that has been missing from the conventional healthcare system. Family and close friends are usually the first ones that notice a change in an individual's behaviors, and yet, their voices could not be given enough weight in mental disorder evaluations because there is no structure in place to seek out as well as to enter family/friend's impressions of the individual's wellbeing. An individual may not present a mental disorder's symptoms in the way expected from the individual's condition due to extensive coping mechanisms at a particular assessment point-in-time. A highly depressed postpartum mom, for example, may look well-groomed if grooming is her coping mechanism of seeking normalcy while an equally depressed individual would most likely lack even basic personal hygiene. People close to the individual, such as family members or close friends, often have a good observation of the individual's struggles. Their insights (as collateral information) can be extremely useful. However, reaching these contacts through conventional methods is an extremely arduous and time-consuming task; hence such collateral information is often not accessed or integrated into mental healthcare diagnosis or delivery systems.

In the present implementations, however, data entries from an individual's circle of friends and family about the individual's well-being is considered as part of the input data for the individual. The implementations use subjective opinions of the people in an individual's circle of friends and family in addition to other inputs received from other resources (e.g., health records, a history of disorders diagnosed for the individual, etc.), to provide an objective evaluation of the individual's health.

The implementations use artificial intelligence models to diagnose or predict an individual's health disorders based on the inputs received about the individual.

Compared to other diagnostic models in this field, the model presented here is a multi-label model that performs concurrent computation of multiple behavioral disorders. In some implementations, the model analyzes the input data at multiple nested layers. In some implementations, the model includes multiple neural networks that are nested together. For example, multiple layers of one neural network are nested to multiple layers of another neural network within the model.

The model is complex even at its input. In some implementations, the model has multiple layers of input neural networks. Each of those input neural networks receives input data related to a particular health marker different from other input neural networks. Each input neural network has an output layer that provides respective probabilities for the same disorders that are considered for the overall model.

The input data is multi-sourced, multi-lateral data. It is multi-source because it is received from multiple sources, e.g., sensors, individual's self-evaluations, friends and family observations. It is multi-lateral because it is received from multiple users of the platform in addition to being received from the target individual's device; examples of those users include member of the individual's circle of friends and family, professionals interacted with the individual in non-clinical off-patient settings, the individual's clinical care providers. The input data includes time-series data, that as explained above, includes the individual's health markers over a specified period of time rather than over single points in time.

The models presented here can be used as agentive tools, or as assistive tools, or both. A model as an agentive tool would provide a stand-alone diagnosis, treatment, and risk assessment without intervention of a clinician. An advanced version of the model can be used to prescribe and manage medication and/or other therapeutics, for example, under supervision at a case by case level or at patient population level.

A model as an assistive tool would help a clinician in diagnosing and assessing severity of pre-specified behavioral disorders. Such disorders can be specified by the clinician, for example, as the most common behavioral disorders. The assistive model would help a clinician in minimizing diagnostic errors, increasing diagnostic confidence, and reducing the time needed to diagnose certain disorders. An assistive model can be beneficial both to a mental health licensed clinician, as well as a non-licensed healthcare provider such as an obstetrician who wishes to diagnose postpartum depression on patients, and possibly, prescribe proper medications to them.

The details of one or more implementations of the present disclosure are set forth in the accompanying drawings and the description below. Other features and advantages of the present disclosure will be apparent from the description and drawings, and from the claims.

### DESCRIPTION OF DRAWINGS

FIG. 1 depicts an example environment that can be used to execute implementations of the present disclosure.

FIG. 2 depicts an example artificial intelligence model according to implementations of the present disclosure.

FIG. 3 depicts example input for the model of FIG. 2.

FIG. 4A depicts an example input neural network pre-processing a set of the raw input data for the model of FIG. 2.

FIG. 4B depicts another example input neural network pre-processing the set of the raw input data for the model of FIG. 2.

FIG. 5 depicts an example process that can be executed in accordance with implementations of the present disclosure.

FIG. 6 shows a schematic diagram of an example computing system that can perform the methods described in the present disclosure.

Likely labeled components in the figures refer to the same elements or steps.

### DETAILED DESCRIPTION

Behavioral health disorders such as depression, obsessive-compulsive disorder, psychosis, etc. are often diagnosed by evaluating the presentation of symptoms that meet certain diagnostic criteria (e.g., DSM-V-TR or ICD-10) at the time of the diagnostic assessment. The assessment is usually conducted in one or two sessions by a licensed mental healthcare provider. Due to lack of timely guidance and triage of in-need individuals, diagnostic encounters are often delayed by months to years from the emergence of symptoms. By the time a person is diagnosed, often their health conditions have deteriorated, leading to complicated, comorbid and/or secondary health conditions, relationship and occupational problems, further complicating the process of reaching the correct diagnosis of the primary health issues.

Accurate diagnostic evaluations are also hard to achieve because the constellation of symptoms considerably vary from person to person and context to context. While an individual with an anxiety disorder may present one of the telltale signs, the same disorder may seemingly not present similar signs in another person suffering from the same disorder. For example, while difficulty in initiating sleep may be a sign of anxiety disorder, a new parent suffering from anxiety may not show any such signs due to extreme sleep deprivation. Chronic sleep deprivation and fragmented night-time sleep due to feeding a baby may mask sleep related symptoms, such as difficulty in initiation or maintenance of sleep.

Implementations of the present disclosure provide techniques for diagnosing and predicting mental health conditions of an individual. The implementations can be used to concurrently diagnose and/or predict multiple behavioral disorders of an individual. Some implementations also recommend respective actions for treating or preventing those behavioral disorders.

Diagnosing a behavioral disorder for an individual means that the individual currently suffers from the behavioral disorder. Predicting a behavioral disorder means that the individual will likely (i.e., with a chance higher than a pre-specified value, e.g., with more than 50% chance) suffer from the behavioral disorder within a predetermined period of time in the future, e.g., starts suffering within the next two years.

The implementations can also be used to determine level of urgency in treating at least some of the diagnosed or predicted behavioral disorders. Depending on the level of urgency, the implementations can send one or more alerts to the individual, friends or family of the individual, emergency contacts of the individual, or healthcare providers such as the individual's doctor, therapist, etc.

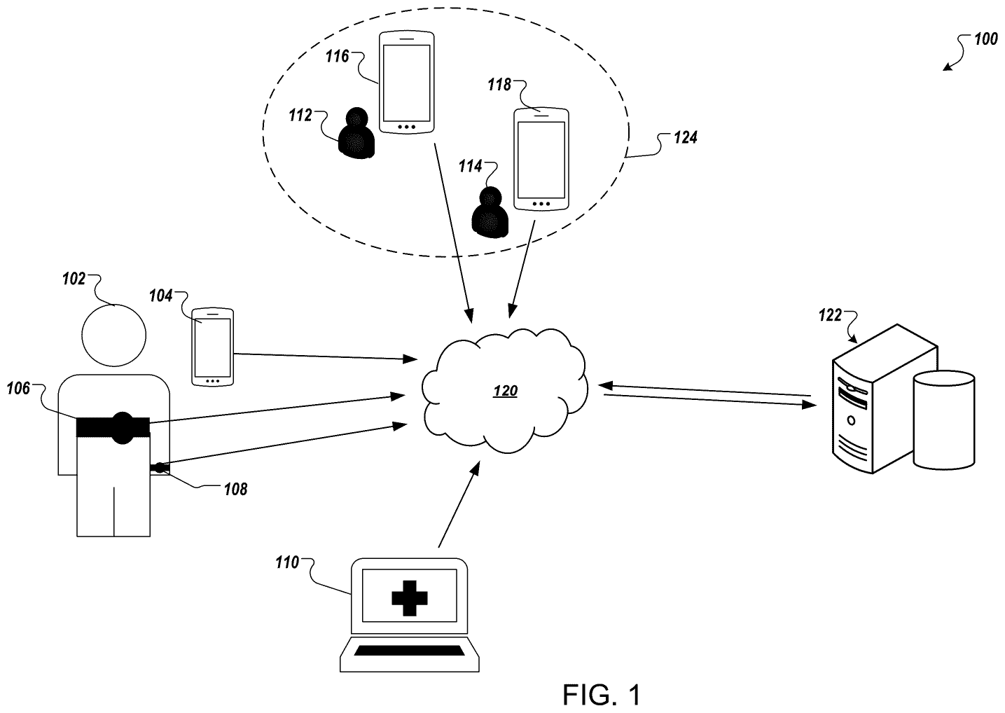

The diagnosis or predictions are achieved by running artificial intelligence models on a computing system. FIG. 1 illustrates an example environment 100 that can be used to execute implementations of the present disclosure. Environment 100 includes a computing system 122 that runs the artificial intelligence models. In some implementations, the models can be executed partly on the computing system 122 and partly on the individual's (e.g., a patient's) computing device.

FIG. 1 depicts an individual 102. Individual 102 can be a patient, or in general, a user of a platform that uses environment 100 to monitor, identify, and/or predict the behavioral disorders of individual 102.

Individual 102 has a user device 104 that the individual 102 uses to access the platform. The individual 102 interacts with user device 104 to use the platform, for example, to enter the individual's health and life information, and to receive information about the diagnosed or predicted behavioral disorder of the individual 102. User device 104 of individual 102 uses the platform to communicate with computing system 122, for example, to send information about the current or past medical and health history of the individual to system 122, and to receive the diagnosed and/or predicted health disorders of the individual from the system 122. The platform uses network 120 to connect the user devices of the users of the platform (e.g., 104, 116, 118, 110) to the computing system 122.

One or more users (e.g., 112, 114) of the platform can be associated with the individual 102 as having specified relationships with the individual 102. For example, individual 102 can identify a circle of his friends and family on the system. The circle includes user 112 as a friend, and user 114 as a family member (e.g., a parent) of the individual 102. In some implementations, there are at least two people different from the individual 102 in the circle of family and friends.

System 122 receives inputs from members of the circle, and uses those inputs to predict or diagnose behavioral disorders of the individual 102. Each user (112, 114) in the circle of friends and family is associated with a respective user device (116, 118) on the platform, and uses their user device to enter their observation of individual 102's life routines, behavioral characteristics, or any warning signs of mental issues that they observe. Since family members and (close) friends are often the first people to recognize signs and symptoms of a developing mental disorder, such observations would play important roles in accurately predicting or diagnosing such disorders. A member of the circle of family and friends can enter their observations by writing down a text on their corresponding user device, by recording a video or audio of themselves explaining their observations, and/or by flagging or selecting an option as answer to one or more pre-specified questions about the individual presented on their device.

Also since close friends and family members are usually the first ones to accurately gauge the crisis risks (e.g., suicides, psychotic episode) associated with the person, system 122 is designed to send alerts regarding existence or such crisis risks to those members of the circle. The alerts can be shown on respective user devices (116, 118) of those users (112, 114) as notifications, for example, being displayed, playing an alerting sound, vibrating the device accompanying a text explaining the alert. Crisis risk as used in this disclosure is any behavioral disorder that would threaten a person's (including the individual's or someone else's) health in danger as a result of the individual's behavior or act. The danger includes, but is not limited to, requiring hospitalization or prolonged treatment of that person as a result of the individual's behavior or act.

System 122 can receive inputs from one or more sensors (106, 108) attached to individual 102, and use those inputs to monitor the individual's routines or disruptions in the individual's routines. The sensors can be attached to the individual's body, e.g., be in touch with the individual's skin, or can be remote from the individual's body, e.g., a camera recording motions. In the depicted example, sensor 106 is worn as a chest belt to monitor, for example, breathing routine, heart rate, etc. of the individual. Sensor 108 is worn as a watch monitoring, for example, blood oxygen level, heart rate, blood pressure, etc. System 122 uses inputs from the sensors for making predictions or diagnosis of the individual 102's behavioral disorders.

In addition, or alternatively, system 122 can receive input data from a healthcare provider of individual 102 for making prediction/diagnosis regarding individual 102. The healthcare provider such as a licensed practitioner, or a non-clinical provider, such as a physical therapist, providing evaluations about individual 102 regarding non-clinical interactions with the individual. User device 110 is depicted as an example device used to receive such healthcare data from users other than the individual 102 and the members of the individual's circle of friends and family.

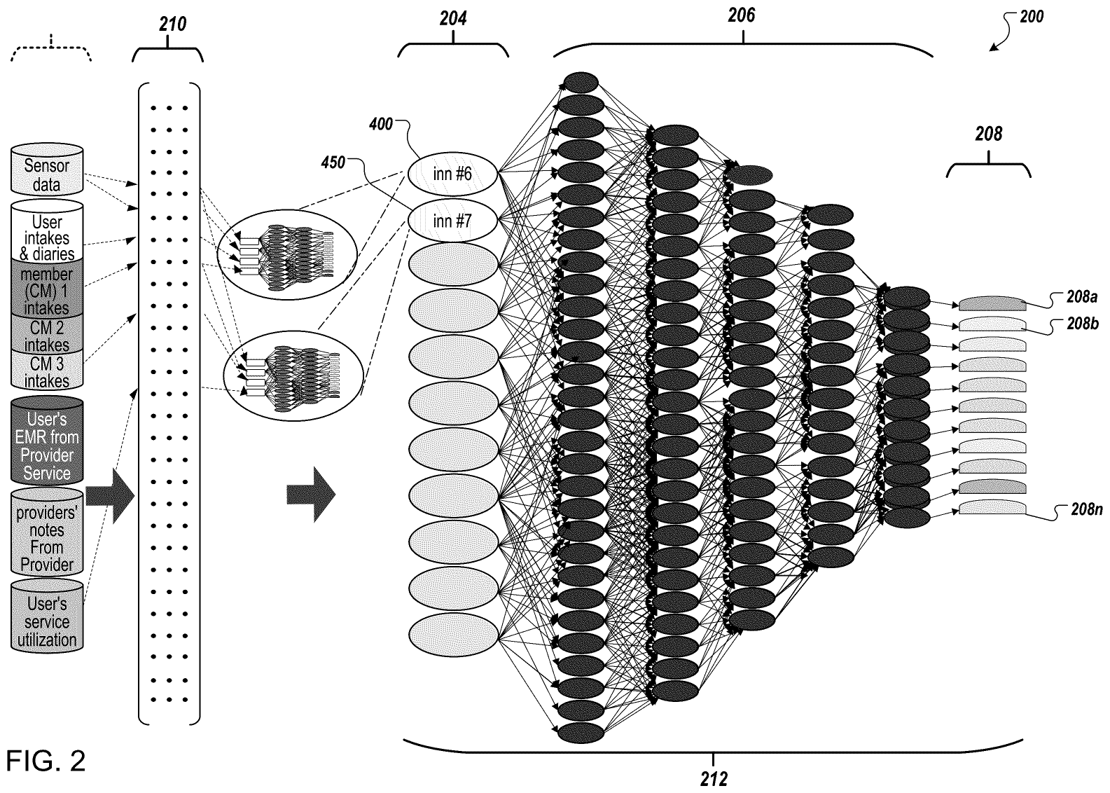

System 122 uses an artificial intelligence model, for example, a machine learning model, to predict and/or diagnose behavioral disorder(s) of individual 102 based on the received input data. FIG. 2 shows an overview of an example artificial intelligence model 200, which system 122 (in FIG. 1) can use to process the raw input data (202) and provide the prediction/diagnosis as output (208).

The model 200 includes a neural network 212 that has an input layer including multiple input modules 204. Each of the input modules (204) runs a respective input neural network (“INN”) on the data provided to it. The model 200 pre-processes (210) raw input data 202, runs input neural networks (“INN”) on the pre-processed input data at input modules 204, and analyzes the output of the INNs through hidden layers 206 of neural network 212 to provide output 208.

Model 200 in FIG. 2 can concurrently predict/diagnose probability of multiple behavioral disorders for an individual (e.g., 102 in FIG. 1). This is an improvement over prior models, where one model could provide an output for only a single disorder. Output 208 includes multiple probabilities (208a, 208b, . . . , 208n) that each indicates the probability of the individual suffering from a respective behavioral disorder in a set of multiple pre-specified disorders. Example behavioral disorders and conditions considered in output 208 are, but is not limited to, suicide attempt risk (with a respective probability provided, e.g., in 208a), risk of a physical harm-inflicting psychosis episode (with a respective probability provided, e.g., in 208b), depression (e.g., in 208n), anxiety disorder, obsessive compulsive disorder (OCD), panic attack disorder, post-traumatic stress disorder (PTSD), bipolar I disorder, bipolar II disorder, psychotic disorder, or other behavioral health conditions.

To predict or diagnose a behavioral condition of an individual, neural network 212 (FIG. 2) analyzes multiple health categories of the individual's health conditions. Each health category is associated with respective health marker(s) that are obtained as part of the pre-processed data (output of 210) through extraction and/or transformation processes performed on the raw input data 202. For example, a “sleep” health category can relate to health markers such as (night time) sleep duration, latency, number of awakenings, causes of awakenings, anxiety about sleep, etc.

Pre-processing module(s) 210 extracts these health markers from raw input data 202. The pre-processing module(s) 210 can also transform the extracted health markers to respective health features that each has a respective format associated with respective module(s) in input modules 204. The pre-processed data (210) is then inputted to input modules 204.

Each input module (204) is associated with a respective health marker. A health category (e.g., health of an individual's children) can include one health marker and be processed by one input module. Another health category (e.g., sleep) can include multiple health markers (e.g., sleep quantity, sleep quality, sleep need, sleep stress), and thus, be processed by multiple input modules. Examples of health categories include, but is not limited to, behavioral health, physical health, management of clinical conditions, trauma experience, reproductive experience, sleep health (e.g., sleep quantity, sleep quality, sleep need, sleep stress), health behaviors, social and connectional health, occupational health, functional health, financial health, genetics and inherited health, societal and structural health, partner health, and children's health.

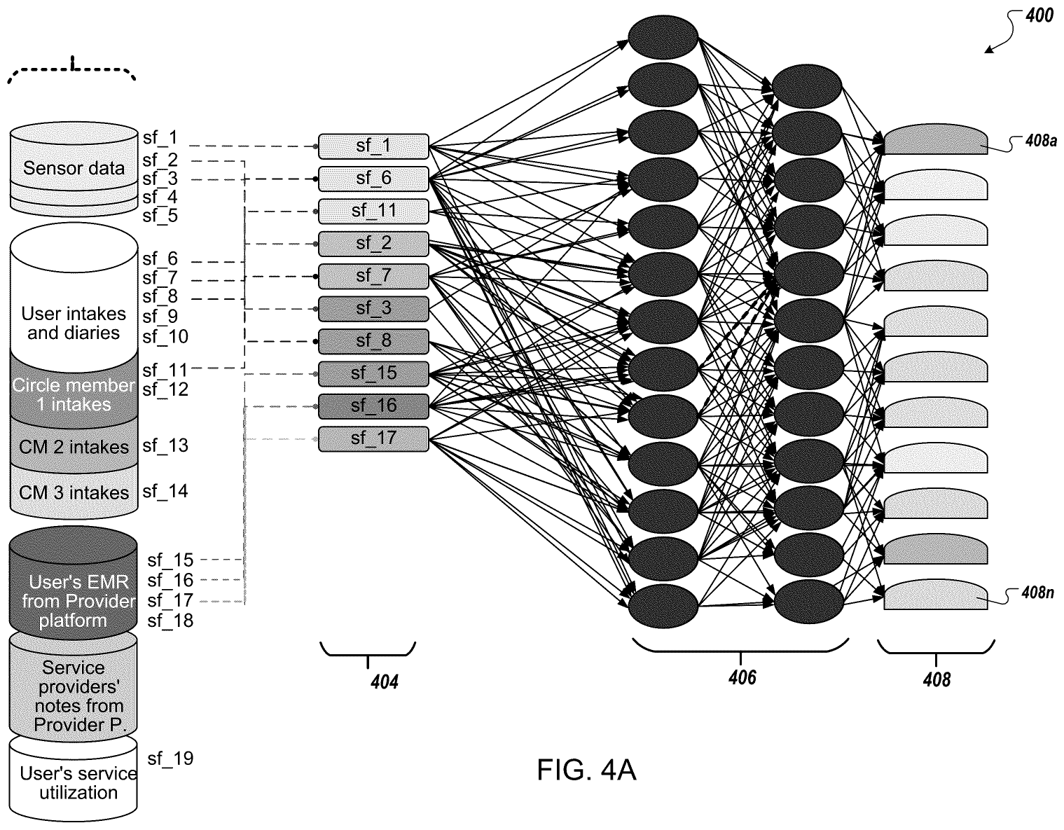

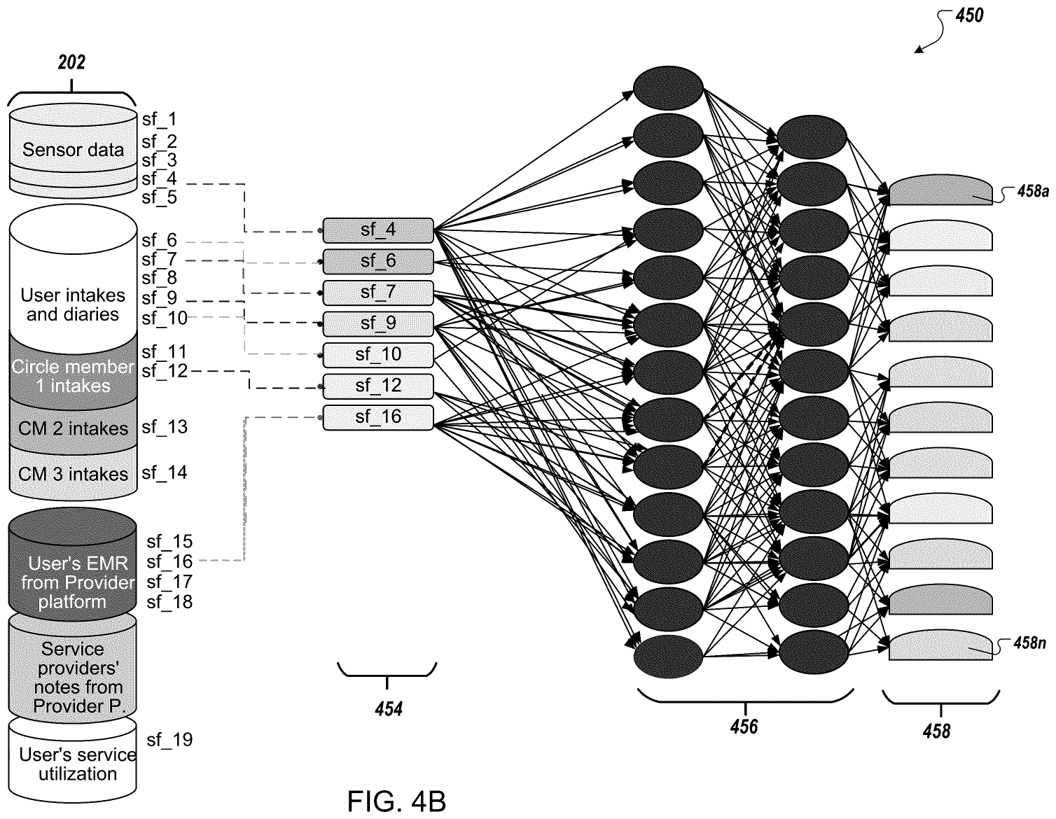

Each of the input modules 204 receives (the pre-processed data including) health features corresponding to the health marker that the module analyzes, and runs a respective INN on those health features. FIG. 2 shows two example input modules 400 and 450 running respective INN #6 and INN #7 on respective pre-processed (data obtained from) raw input data 202. More detailed views of input modules 400 and 450 are depicted in FIGS. 4A and 4B, respectively. Module 400 corresponds to health marker sleep quantity, and module 450 corresponds to health marker sleep quality.

In FIG. 4A, module 400 uses pre-processed data 404, which includes sleep features (“sf”) as health features extracted and transformed from raw input data 202. Since both sleep quantity and sleep quality are health markers of the same health category (i.e., sleep), the two modules 400 and 450 can share certain health features. However, as can be seen from FIGS. 4A and 4B, each of the modules 400 and 450 uses a different set of such sleep (health) features, 404 and 454, respectively. In addition to sleep features 6, 7, and 16 that are used by both modules 400 and 450, module 400 uses sleep features 1, 2, 3, 8, 11, 15, and 17, that module 450 does not use; and module 450 uses sleep features 4, 1, 10, 12 that module 400 does not use. For example, both modules 400 and 450 can use the sleep feature of number of awakenings (sf_6). Module 400, which is regarding sleep quantity health marker, can also use the number of naps taken during the day (sf_1). Module 450, which is regarding sleep quality, can use breathing rhythm (sf_4) sleep feature for the time that the individual is asleep.

As noted earlier, each input module (204) runs a respective input neural network (INN) on the received health features. In FIG. 4A, hidden layers 406 of the INN run on the health features obtained for module 400. In FIG. 4B, hidden layers 456 of the respective INN run on the health features 454 obtained for module 450.

The result of an INN's data processing would be one or more output values provided in the respective output layer of the INN. An output layer can provide multiple outputs that each represents a respective probability of a behavioral disorder. The INN in module 400 in FIG. 4A, for example, has output layer 408 providing outputs 408a through 408n; and the INN in module 450 in FIG. 4B has output layer 458 providing outputs 458a through 458n.

The output layer of each INN provides probabilities associated with the same pre-specified behavioral disorders that are analyzed by the overall neural network (212 in FIG. 2) of the model 200. For example, if the overall neural network 212 is analyzing data for three pre-specified behavioral disorders, each of the output layers 408 and 458 of INNs #6 and #7 running in respective input modules 400 and 450 would also provide probabilities for the same three pre-specified behavioral disorders.

However, each input module (e.g., 400, 450) is associated with only one respective health marker, and predicts/diagnoses for those three pre-specified behavioral disorders by considering only health features associated with that respective health marker. This is while the overall neural network (212) predicts/diagnoses for those three pre-specified disorders based on health features extracted for a plurality of health markers (that each is evaluated by a respective input module). As noted earlier, the hidden layers (206) of the overall neural network 212 predicts/diagnoses for those three pre-specified behavioral disorders by analyzing the outputs of input modules 204, which are associated with different health markers.

For example, if the main neural network 212 determines the probabilities of an individual suffering (currently or in the future) from behavioral disorders of depression, psychosis, and bipolar I, each of modules 400 and 450 also determines the probabilities of suffering from those three disorders, but by just considering the respecting sleep features used by INNs #6 and #7, respectively. Input module 400 that is about sleep quantity, can provide the probability of an individual suffering from depression, the probability of the individual suffering from psychosis, and the probability of the individual suffering from bipolar I disorder by analyzing sleep quantity features 404. Input module 450 that is about sleep quality, can provide respective probabilities of an individual suffering from depression, psychosis, and bipolar I disorder, by analyzing sleep quality features 454. The (probability) outputs of the input modules are then analyzed through hidden layers 206 of the overall neural network 212 to provide the final probabilities of those three behavioral disorders for the individual, as the output layer 208.

Accordingly, the probabilities calculated at an input modules can be calculated independently from the probabilities calculated at another input module, but the probabilities calculated for all modules are used by the hidden layers 206 in determining the final probabilities provided at the output layer 208 of the overall neural network model (212). This method enables prediction based on individual categorized health markers, in addition to considering all health markers as a whole. By doing so, one can learn (i) which health markers are more predictive of which behavioral disorder (e.g., sleep quality or quantity for psychosis or for depression), and (ii) which health features (e.g., subjective entry of sleep duration by the individual or a watch sensor's measurements of the sleep quality) are more reliable, with more accurate and minimal human-involved classification at the module level.

Alternatively, or in addition, a human can label data for each or some of the input modules, and the modules can be used to predict that module's health-marker's normalcy (i.e., healthiness). For sleep quantity module 400, for example, a sleep expert may label health features to distinguish normal/healthy sleep versus problematic sleep, and train the module based on the labeled data.

In some implementations, the outputs of the input modules and/or the outputs of the model can be scores instead of probabilities in accordance with the training datasets. Score thresholds can then be set based on the score distributions observed. For example, scores lower than a first threshold (e.g., the score at 50th percentile) may be assigned to a first label (e.g., no-case, low-risk, etc.), scores higher than the first threshold and lower than a second threshold (e.g., the score at 50th percentile and the score at 80th percentile) may be assigned to a second label (e.g., mild-case, elevated risk, etc.), and scores higher than the second threshold (e.g., the score at 80th percentile) may be assigned to a third label (e.g., severe-case, high risk).

The threshold(s) can be pre-specified. Each threshold can be pre-specified based on the targeted behavioral disorder. For example, on a system, depression (as a first behavioral disorder) can be assigned to five different thresholds, while bipolar I is assigned to only three different threshold. As noted above, the scores found between two consecutive thresholds is assigned to a respective label.

In some implementations, the outputs of the input modules 204 are nested through hidden layers 206. For example, output layers of multiple input modules can interact, or be connected to each other in a hierarchical, ensemble, or stack form.

In some implementations the nesting can happen in other layers, for example, in addition to the nesting of the output layers of the INNs. For example, one or more of the hidden layers 406 in FIG. 4A can be nested with one or more of the hidden layers 456 of module 450 in FIG. 4B. In some implementations, multiple modules are connected with each other in an hierarchical organization, ensembling and/or stacking, creating a nested network model that considers all alternative behavioral health disorders from among the pre-specified disorders considered by model 200.

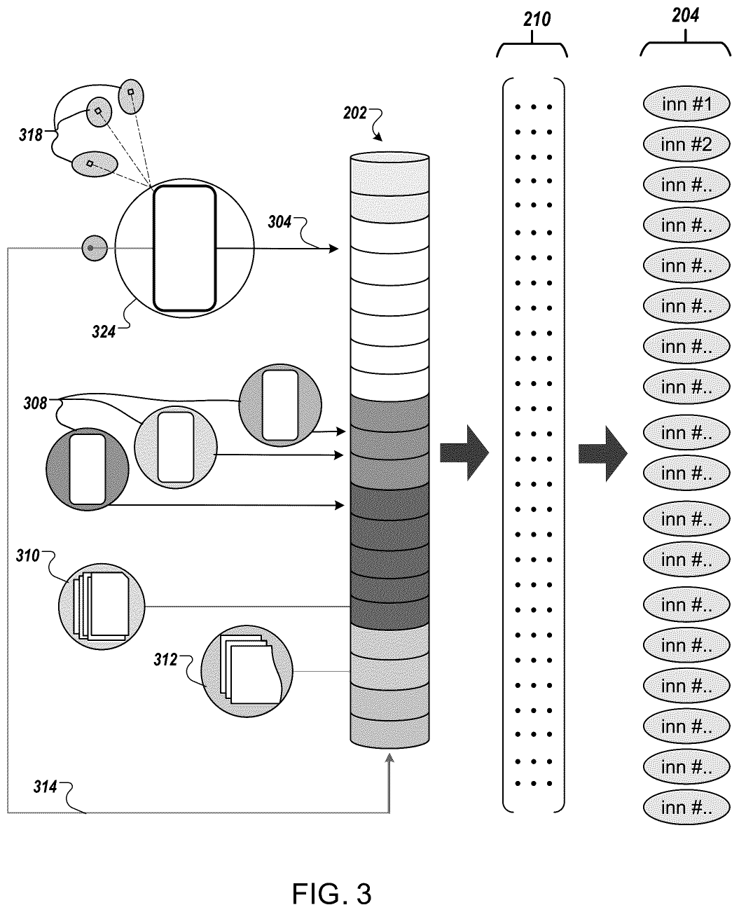

As explained above, raw input data 202 is pre-processed before entering the input modules 204 (FIG. 2). FIG. 3 shows an overview of pre-processing data (210) before entering input modules 204. The pre-processing module(s) 210 pre-process raw data 202 to extract health features, to transform data modalities, and/or to cleanse the raw data 202 so that the data would be ready for entering the respective input modules 204.

The health feature extraction is module-specific. This means that for each module in input modules 204, only health features are extracted that correspond to the health marker associated with that module. For example, for module 400 associated with sleep quantity, only sleep features that associate with sleep quantity are extracted; for example, sleep duration, starting time of sleep, number of awakenings during a respective sleep episode, causes of awakenings, sleep anxiety, heart rate during sleep, etc. For another module corresponding to children's health, only features indicative of or related to the individual's children are extracted from the raw data; for example, interactions with children, hours spent with children over a particular period of time, children's health history, children's school records, etc.

The modality transformation can depend on the input data format, but can also be module-specific. As explained above, the example raw input data 202 can be obtained from a variety of sources, and thus, can have multiple modalities. For example, in FIG. 3, raw input data 202 is received from an individual's user device 324, from user devices 308 of people in the circle of friends and family of the individual, from healthcare sources such as clinical healthcare provider 310, or non-clinical healthcare provider 312.

The data received from the user device 324 itself can also have a variety of modalities (or formats) as it can be received from different sources that generate data in different modes or formats. For example, user device 324 can receive data from sensors 318 (which each of them can generate data in a modality different from other sensors), from individual's inputs such as diaries, answering questionnaires, from the individual's usage of the user device 324 and the applications used on the device, etc. Alternatively, or in addition, one or more of the sensors 318 can send their generated data directly to system 122 instead of communicating to system 122 through user device 324. However, such implementation would require more power for the sensors as the sensors would likely need to send their data a farther distance and through busier traffic than in the first configuration where the sensors can use a local network to communicate their data to the user device 324. Example modalities for the input data includes structured text, categorical or numerical data, unstructured narrative text data, video, audio, sensor data, data from connected devices and phone APIs, biological signals from clinical or nonclinical measurements, user device and certain device application uses, etc.

Accordingly, through the preprocessing, each health feature extracted for a module can be transformed to a respective format (or mode) associated with that module. For example, a video received as raw data can be transformed into an emotional state metric for an anxiety module. As another example, an audio file can be transformed into text for sentiment analysis for a depression module. At least two pieces of raw input data (202) can have different formats. The formats can include one or more of voice, audio, text, an option selected on a user device (e.g., 116, 104 in FIG. 1).

Since each module receives and analyzes the data in a respective format, the same health marker (extracted from input data 202) can be transformed to multiple health features for use in different input modules. For example, both input module 400 (associated with sleep quantity) and anxiety module 450 can use different formats for awakenings at night. Episodes of awakening at night can be transformed to a number (e.g., five) indicating fragmented sleep for sleep quantity module 400, while the same data can be transformed to a binary format (e.g., zero or one) for an anxiety module to indicate the presence of night-time anxiety. In some implementations, at least two input modules are associated with different formats. As explained above, in some cases, at least one extracted health feature is transformed to the different formats associated with the at least two modules.

Pre-processing module(s) 210 can also cleanse data to reduce noise. Raw input data 202 can have noises and irrelevant information that may not play a significant role in prediction/diagnosis of the individual's behavioral disorder. For example, a ten page report about a conversation between the individual and a family member may be summarizable into a sentence. Or sensor data about a night long sleep may be summarizable into a report of a few sentences. Through pre-processing, the raw data is cleansed to remove the noises, and/or to extract health features that would indeed play roles in the disorder prediction/diagnosis. The cleansing process would reduce the input data volume and thus, reduce the processing time and increase the processing speed for the neural network (e.g., 212). The cleansing process can happen before, during, or after any of the health feature extraction and transformation processes.

As explained above with respect to FIG. 2, the overall neural network 212 has an output layer 208 that provides respective probabilities of an individual suffering or expected to suffer from multiple pre-specified behavioral disorders. In some implementations, those probabilities are analyzed to determine an urgency in treating at least one behavioral disorder in the pre-specified disorders. Such analysis can be performed by a particular module of model 200 (not shown), by a separate module that receives outputs from model 200, or by a human operator.

In some implementations, the urgency is determined in terms of the probability that the individual would commit suicide, commit infanticide or hurt child or another person other than self, experience a psychotic episode or manic phase of a bipolar disorder, cause irreparable harm to their familial, social, professional or occupational relationships or loss of financial status, be harmed by a diagnostic-error (such as a bipolar individual being misdiagnosed with unipolar depression and treated with an anti-depressant that then triggers a manic episode).

If it is determined that the urgency exists for the target individual (e.g., 102 in FIG. 1), the computing system 122 (FIG. 1) can generate and send an alert to at least one of a healthcare professional, an individual in the circle of friends and family, an emergency contact, etc. of the target individual or the individual to notify them about the urgency. In some implementations, an urgency in treating a particular behavioral disorder exists if the determined probability (associated with the urgency) is higher than a pre-specified threshold value. The pre-specified threshold value can vary based on the behavioral disorder. For example, the threshold value for committing suicide can be set to 0.2, while the threshold value for causing loss of financial status can be set to 0.7.

In some implementations, computing system 122 generates and sends out a status alert regarding the probabilities calculated for the target individual's behavioral disorders. The status alert would include more information than a general notification of the existence of an urgency. For example, the status alert can include information about the state of each behavioral disorder detected for the individual, such as prodromal, mild, moderate, severe. The status alert can include recommendations on treatment actions to take, such as particular exercises or diet routines, particular medications for respective identified behavioral disorder(s) (for example, the one(s) that the system found to need urgent intervention). The status alert can include action(s) to arrange, triage, or initiate healthcare utilization or other services for diagnosis, treatment, treatment supplement, or pain relief for the identified behavioral disorder(s).

In some implementations, system 122 can compare the output of the model 200 to a medical history of the individual (102) to detect worsening or continued state of undesirable symptoms or unsustainable side-effects of interventions in the disordered state. The system can recommend additional or different actions compared to what was recommended before or compared to the treatments and actions the individual has been taking. Such recommendations can include medication management, physical exercises, diet or sleep routines, contact of one or more healthcare professionals specialized in treating respective behavioral disorders, etc.

In some implementations, system 122 can compare the output of the model 200 to a medical history of the individual (102) to detect improvements or continued state of desirable stabilization of symptoms in a disordered state. The system can recommend continuation of or addition of other actions compared to what the individual has been taking to facilitate further improvements.

In some implementations, the system 122 can detect prodromal symptoms. The system can recommend proactive actions to provide symptom relief, improvement, and/or prevent development of the disorder into clinical state.

In some implementations, the system 122 can detect normal state of health (i.e., complete euthymia), for example, by determining that the individual is free of clinical or prodromal symptoms. The system can recommend actions to facilitate the continuation of euthymia, or prevention of such symptoms for one or more of the pre-specified behavioral disorders.

In some implementations, the system 122 can detect the probability of experiencing specific symptom groups such as depressive symptoms, anxiety related symptoms, symptoms of suicidality, psychotic symptoms, obsessive symptoms, etc., instead of the probability of having clinical disorders in order to allow trans-diagnostic, system-based evaluations and treatment management. Providing probabilities of the symptoms can help a health practitioner or patient to focus on particular symptoms and monitor any changes over the time. This can improve accuracy in diagnosing and/or treating the underlying health disorder(s).

The computing system 122 can send any of the recommendations and/or status alerts noted above in case of determining that an urgency exists and just for the respective disorders determined to have urgency, or in general, with or without determining the urgency. In addition, or alternatively, system 122 can generate a report of the health status of the target individual, including the respective diagnosis or a prediction of the pre-specified multiple behavioral disorders. The report can include respective probabilities of the target individual suffering from the multiple behavioral disorders, and/or respective levels of urgency in treating one or more of those disorders.

Each of the overall neural network model 212 and INNs of respective input modules 204 can be trained for diagnosing or predicting the pre-specified mental (or behavioral) disorders. The training can include using health histories of a plurality of training individuals including (i) disorder-suffering individuals who suffer from one or more of the pre-specified mental disorders, and (ii) normative individuals not suffering from the at least one of the pre-specified mental disorders. In some implementations, the training for a particular behavioral disorder includes using an equal (or representative, e.g., proportional) densities of normative and disorder-suffering individuals. Representative densities can be specific to the targeted disorder. For example, depression (which happens to 1 in every 5 individuals) is much more common than psychosis (which happens to 1 in every 1000 individuals). For representative densities, the samples can correspond to the proportional cases observed in real life instead of using equal number of normative and disorder-suffering individuals.

As explained above, inputs to model 200 are received from a variety of sources that are associated with the individual 102. Referring back to FIG. 1, some of such sources are the individual 102, members of circle of friends and family (112, 114) of the individual, a healthcare provider of the individual, sensors (106, 108) attached to or sensors that monitor the individual during the individual's daily life.

The sources of the input information can use a platform running on the respective user devices of the users of the platform to enter information about individual 102. For example, individual 102 uses user device 104 to enter information such as writing a diary, selecting or writing down certain answers to questions asked on the platform, taking videos or images, recording audio, etc. to the platform. Similarly, members of the individual's circle of friends and family 112, 114 can use their respective user devices 116, 118 to enter their observation of the individual 102 to the platform.

In some implementations, a user interacts with the platform through an application running on a user device of the user. The user of the user device interacts with the platform, for example, by entering information on the application, e.g., by answering certain questions about the individual. Alternatively, or in addition, the platform can be presented to a user in the form of a web-page.

A user can set up their circle of friends and family on the platform by inviting a certain number (e.g., three) of the individual's friends or family members to the platform. The user can use, for example, a mobile application to log into their account on the platform, to enter data, and/or to make any changes to their profile on the platform. Each member in the circle of friends and family can use the application similarly to the way that the individual uses the application. But if any of the members want to sign up for an event (e.g., a paid event) or invite other users to their own circles, they, too, may need to subscribe to the platform, for example, by paying a fee to the platform.

Each subscriber of the platform and their connected circle member form a dyad on the platform. Members of a subscriber's dyad can provide input on the subscriber's health and wellbeing through completing prompted or unprompted intakes. In some implementations, a subscriber's dyads receive emergency alerts about the subscriber's health in the case of a crisis risk. A subscriber can change their circle, for example terminate a dyad, replace a certain member in the circle with someone else, or add to the members of the circle.

The platform enables a subscriber to search through, pay for, receive services during or participate to a variety of non-clinical events. The event can take place online. An event can have one or more hosts that would observe the participants. Based on a participant's interactions during these events, a host can put a flag on the participant's profile if the host is concerned about the participant's wellbeing (e.g., elevated crisis risk). This flag can be used as an additional collateral input on the participant's individual mental/behavioral health evaluation.

The platform is a single tool for bringing individuals, their circle members and care providers together. The individual and their circle members can use an application, e.g., an iOS application, that is used (1) as a behavioral monitoring module that collects a variety of input data, and provides, e.g., displays, intakes and alerts, (2) as a psycho-educational module that publishes targeted content, and/or (3) as a marketplace where the user can search among available providers and clinical and nonclinical services, schedule, join and pay for them.

Care providers can use the platform 1) as a practice support suit that works as a scheduler, marketplace and a payment processor, 2) as a clinical support tool that provides access to clients' data and predictive analytics, and/or 3) as an integrated electronic medical records (EMR) where providers can report and read all prior clinical reports on their clients.

In some implementations, depending on the source, type and the modality of the data, data collection is either triggered automatically on pre-set intervals (e.g., connected sensor data, user engagement data) or triggered by the user submissions (e.g., user intakes, health provider reports). For example, questions are asked to the user of the platform periodically. The period can have a default value, e.g., a week, or can be set by the platform based on the user's condition, or the period can be variable and the data entry is determined by the user of the user device, or by the individual who has identified the user of the user device as a member of the individual's circle of friends and family.

In some implementations, a sensor (either as attached to the individual 102, separated from the individual 102) that measures or monitors one or more health or lifestyle parameters of the individual, can also communicate with the computing system 122. Such communication can be directly, e.g., through network 120, or through the individual's user device 104, e.g., through an application related to the sensor that interacts with an application running by the platform on the user device 104. Examples of the sensors include, but are not limited to, an activity tracker, a sleeping tracker, a blood pressure monitor, or an electroencephalogram.

A user device that uses the platform can be a stationary, or a mobile user device. Examples of a user device include, but are not limited to a computing machine, a cellular phone, a smart phone, a smart watch, a tablet, etc. A user (e.g., 102, 112, 114) can interact with the platform through audio, video, or manual entry (e.g., by hand) of information on the user device of the user.

As noted above, system 122 extracts health features from information received as raw data 202 from one or more user devices. Health features can be extracted from at least one piece of input data based on an analysis of one or more of word-choice, number of words used, repeated words, completion duration, keyword analysis, time of entry, pitch analysis, pace of talk, intonation in the at least one piece of input. Such form of health feature extraction processes would be particularly useful for information (or pieces of input data) received from the individual 102 themselves because (a) it is usually easier for people with behavioral disorder to talk than to write or to manually enter data on a user device, and (b) such form of analysis can identify mental or mood state of the individual from the individual's words or gesture in an audio or video recording in addition to, or instead of, extracting such information from the actual words stated.

In some implementations, computing system 122 receives the raw input data over a period of time, and analyzes those data as temporal data received over a period of time. The period of time can be fixed, or can be pre-specified for particular raw data. For example, computing system 122 may use the individual's sleep routine data as aggregated over a period of at least a week, and use the individual's socialization routines as aggregated over a period of one month. In some implementations, a health feature extracted from one or more pieces of raw input data includes a temporal health pattern (e.g., socializing routines, time of sleep, sleep periods, etc.) of the individual.

In some implementations, system 122 performs a periodic analysis of the input data regarding individual 102, and provides a periodic evaluation (including diagnostics and prediction) of the individual's mental state (including behavioral disorders). In some implementations, system 122 provides a continuous evaluation of the individual's mental state. Upon receiving a new piece of input about the individual 102, system 122 re-runs model 212, and updates its outputs (208) for the individual.

As noted earlier, each of the overall neural network 212 and input neural networks (INNs) 204 can be a multi-layer and/or nested model that can run on multi-modal, multi-lateral, and/or time-series data. One or more of the neural networks can be regression-based, e.g., have recurrent regression structures. One or more of the neural networks can be nested deep learning models. Other structures can be used for any of the neural networks disclosed here, as long as the overall purpose of using that neural network is met. For example, random forest, gradient boosted decision, etc. can be used as at least part of one or more neural networks. In some implementations, the number of layers in the input neural networks are limited to a certain threshold. In some implementations, the number of layers and number of nodes in each layer are determined through examining the model performance across different iterations.

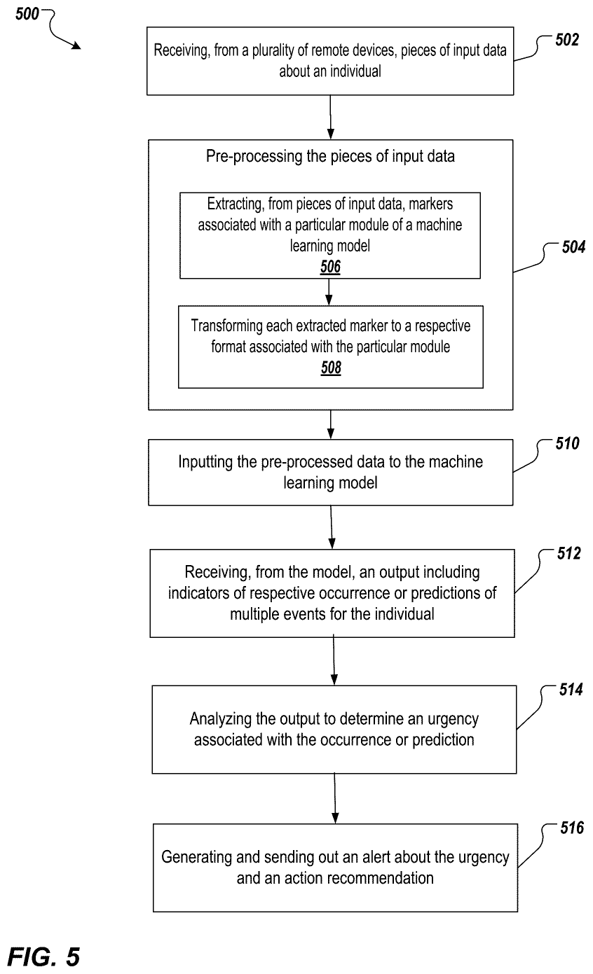

FIG. 5 depicts an example process 500 that can be executed in accordance with implementations of the present disclosure. Process 500 can be performed by a computing system, e.g., system 122 in FIG. 1.

At 502, the computing system receives, from a plurality of remote devices, pieces of input data about a target individual, e.g., individual 102 in FIG. 1. Each piece of input data is received from a respective remote device through a network, e.g., network 120 in FIG. 1. One or more devices in the plurality of devices can be respective sensors (e.g., 106, 108) measuring or monitoring respective health-related parameters of the target individual. One or more devices (e.g., 116, 118) in the plurality of devices can be associated with respective people (e.g., 112, 114) in a circle of individuals that each has a respective specified relationship with the target individual. At least a piece of input data is received in response to an interaction of a person with a respective device in the plurality of devices. The person can be in the circle of individuals (e.g., circle of friends of family 124 in FIG. 1). The person can be the target individual (e.g., 102) interacting with their own device (e.g., 104). Examples of the input data are shown in FIG. 2 as raw input data 202.

At 504, the computing system pre-processes the received pieces of input data. The pre-processing can be performed by a pre-processing module (e.g., 210 in FIG. 1). The pre-processing can include data extraction and transformation to obtain features (e.g., health features) in respective formats suitable for being inputted to a machine learning model (e.g., neural network 212 in FIG. 2) of the computing system. The machine learning model can include multiple input modules, and each input module can be associated with a respective marker (e.g., health marker). For example, input module 400 in FIGS. 2 and 4A is associated with sleep quantity health marker, and input module 450 in FIGS. 2 and 4B is associated with sleep quality health marker.

At 506, the computing system pre-processes the input data to extract markers (e.g., health marker) associated with a particular module of the machine learning model. For example, in FIGS. 4A and 4B, the raw input data 202 is pre-processed to extract health features (i.e., sleep features) associated with sleep quantity and sleep quality, respectively.

At 508, the computing system transforms each of the extracted (health) markers to a respective format associated with the particular module. In some implementations, at least two input modules are associated with different formats. In some implementations, at least one extracted health feature is transformed to the different formats associated with the at least two input modules.

At 510, the computing system inputs the pre-processed data (i.e., the transformed health markers) to respective input modules of the machine learning model. In FIG. 2, the machine learning mode is shown as a multi-layered neural network 212, where the input modules 204 make the input layer of the neural network 212. Each input module, itself, has an input layer including the transformed health features obtained through the pre-processing. In FIG. 4A, for example, input layer 404 of the input layer 400 includes the transformed health features associated with the module's health marker, sleep quantity. An input layer of a first module can be different from an input layer of a second module. For example, input layer 404 of module 400 in FIG. 4A is different from input layer 454 of module 450 in FIG. 4B.

As explained above, each of the input modules can run a respective input neural network (INN) on the input layer of the module. For example, see description of FIGS. 4A, 4B above. The overall neural network 212 analyzes (e.g., through hidden layers 206 in FIG. 2) the outputs of the input modules to provide outputs.

At 512, the computing system receives the output from the model. The output includes indicators of respective occurrence (e.g., diagnosis) or predictions of multiple events (e.g., health-related events such as behavioral disorders) for the target individual. In FIG. 2, for example, output layer 208 includes multiple values that each indicates the probability that the target individual suffers or will suffer (in the future) from a respective behavioral disorder. For example, output 208a can indicate a depression probability, output 208b can indicate an anxiety probability, etc.

At 514, the computing system analyzes the output to determine an urgency associated with the determined occurrence or prediction of events, e.g., an urgency in treating at least one of the behavioral disorders. The urgency for treating a particular behavioral disorder can exist if the probability determined as an output for that particular behavioral disorder is more than a pre-specified threshold.

At 516, the computing system generates and sends out an alert about the urgency. The computing system can also make recommendations on actions to take with respect to the urgency, e.g., about how to treat, alleviate pain, and/or slow down progress of the behavioral disorder. The computing system can send the alert and/or the recommendation to the target individual, to an individual in the circle of friends and family of the target individual, to an emergency contact, and/or to a healthcare professional of the target individual.

In addition, or alternatively, the computing system can generate and send a status alert to at least one of the target individuals, a healthcare professional, or an individual in the circle of friends and family, or an emergency contact of the target individual. The status alert can include information about a detected state of the target individual. The status alert can indicate how urgent it may be to treat a disorder at the detected state. The status alert can include recommendations on actions to take, for example, engaging psycho-educational materials, initiating unlicensed or licensed care services, etc.

As explained above, the pre-processing actions to be taken on a piece of input data depends on the source, type, and/or modality of the piece of input data. For example, for a sleep quality input module (400) or for a sleep quantity input module (450), input data can be pre-processed to extract sleep features such as total night-time sleep duration (e.g., in seconds), sleep onset latency (e.g., in seconds), number of awakenings in a certain episode of sleeping, max duration of a consolidated sleep episode (e.g., in seconds), average duration of a sleep episode, causes of awakenings (e.g., categorical data converted into a numerical scale to represent unfortunate-to-problematic nature of awakenings such as fire-alert in the building activating vs waking up from a night terror), total daytime sleep (e.g., in seconds), daytime sleepiness and fatigue (e.g., categorical data representing none-to-extreme scale of the severity), behind-the-wheel sleepiness and fatigue (e.g., categorical data representing none-to-extreme scale of the severity), subjective account of difficulty in falling asleep (e.g., categorical data representing easy-to-difficult scale of experienced difficulty in initiation of sleep), subjective account of difficulty in sleep maintenance (e.g., categorical data representing easy-to-difficult scale of experienced difficulty in initiation of sleep upon awakening), subjective account of sleep quality experienced the next day (e.g., categorical data representing a refreshing-to-restless sleep quality upon awakening).

In some implementations, most of the sleep data collected will be over a long time period (e.g., over a period of months). This is to capture changes in the individual's sleep patterns or life routines. Example tools that can be used to uncover and analyze such patterns or routines include, but is not limited to moving window statistics (by aggregating values over a rolling window), lagged variables (by incorporating previous time series values as features), time-based features (e.g., by adding day of the week, etc.), autoregressive integrated moving average (ARIMA), or Long Short-Term Memory (LSTM) recurrent neural network.

As explained above, the model (e.g., 200) is trained by using training data. The training data can be obtained from different sources, e.g., from clinical evaluations, from healthcare utilizations (e.g., psychiatric emergency care received, for example, from device 110 in FIG. 1), clinical evaluations, crisis related interactions (e.g., during wellness checks by the police, wellness checks by a neighbor, standardized screener results).

The training data can be labeled by a professional, for example, by a licensed healthcare provider. In an example, two labels can be used: positive and negative. For example, a two level evaluation of a major depression can have a positive label—indicating that at the x time of the appointment, individual Y had clinical depression—and a negative label-indicating that at the x time of the appointment Y did not have clinical depression.

However, more than two labels can also be used, for example, to provide a more granular clinical evaluation. For example, a six level evaluation of major depression can be labeled by: severe depression, moderate depression, mild depression—i.e., clinical diagnosis of depression with mild severity, —elevated risk for depression—i.e., depression related symptoms are present but not yet at a level to pass the diagnostic threshold, —some risk for depression, minimal risk for depression-no signs of depression—at the x time of appointment.

Example health markers that can be used in the model are:
Behavioral health history such as
   Current condition (e.g., within the past 6-months until now),
   Recent conditions (e.g., between the last 2 years and 6-months prior),
   Near-past conditions (e.g., between the last 5 to 2 years),
   Past conditions (e.g., between the last 5 years and 17 years of age),
   Childhood conditions (e.g., during 0-17 years),
Physical health history such as
   Current condition (e.g., within the past 6-months until now),
   Recent conditions (e.g., between the last 2 years and 6-months prior),
   Near-past conditions (e.g., between the last 5 to 2 years),
   Past conditions (e.g., between the last 5 years and 17 years of age),
   Childhood conditions (e.g., during 0-17 years),
Management of clinical conditions such as
   Current medications (e.g., prescribed medications, medication adherence, medication side-effects, value to the user such as benefits, side-effects and hurdles),
   Past medications (e.g., prescribed medications, medication adherence, medication side-effects, value to the user such as benefits, side-effects and hurdles),
   Healthcare utilization (e.g., type of care such as routine care, specialty care, frequency of care, satisfaction of care),
Trauma experience including
   Type of trauma such as
      Interpersonal trauma (e.g., sexual trauma, non-sexual physical trauma, verbal trauma), perpetrator trauma (e.g., from partner, family member, acquaintance, someone from the community, stranger),
      Medical trauma (e.g., excluding birth trauma)
      Birth trauma (e.g., medically induced birth trauma,
      non-medical birth trauma)
      Traffic related trauma,
      Disaster related trauma (e.g., trauma related to nature-related disasters, including human-contributed natural disasters),
      Human-made mass casualty trauma (e.g., trauma related to human-caused, mass casualty events)
      Other trauma
   Type of exposure, such as
      Direct recipient,
      Observant,
      Other (e.g., accomplice-facilitator aggressor experience, aggressor experience),
   Frequency of exposure, e.g., one-time, repeated,
   Timeline of experience, such as
      Fresh experience (e.g., within the past year),
      Recent experience (e.g., between the last 5 and 1 years),
      Prior experience (e.g., between the last 5 years and 17 years of age),
      Childhood experience (e.g., during 0-17 years of age)
   Severity at the time, e.g., subjective, mutually exclusive categorization including
      Life altering severity (if any of the below)
         Created fear for one's life or severe injury,
         Caused any duration of in-patient care,
         Affected any area of the daily function (e.g., sleep problems, occupational problems, change of routine) most days (e.g., 4 days or more per week) for a long period of time (e.g., for more than 6-months),
         Resulted in any type of outpatient or non-clinical (e.g., peer-run support group participation) trauma-related healthcare utilization for a long period of time (e.g., for more than 6-months),
      Debilitating, meaning anything other than life-altering,
   Severity now, e.g., subjective, mutually exclusive categorization including
      Extremely severe (e.g., affects any area of the daily function most days of the week, e.g., 4 days or more per week),
      Severe (e.g., affects any area of the daily function some days of the week, e.g., 2-3 days per week),
      Considerable (e.g., affects any area of the daily function some days of the month, e.g., 0-1 days per week, 1-7 times per month),
      Latent (e.g., affects any area of the daily function some days of the year, e.g., 0-1 days per month, 0-12 times per year),
Reproductive experience, such as
   Conception-related,
   Pregnancy-related (e.g., miscarriage, abortion, late-term loss, stillbirth
   Labor & birth-related (e.g., infant loss during labor, birth or before discharge from the hospital),
   Postpartum-related (e.g., infant loss during the first year of life),
Sleep-health, such as
   Sleep quantity
   Sleep quality
   Sleep need
   Sleep stress
Health behaviors, such as
   Substance use
      Type of substance (e.g., tobacco, marijuana, alcohol, opioids, amphetamines, prescription medication),
      Frequency of use,
      Duration of use,
      First time of use,
      Abstinence history
   Activity
      Lifestyle (e.g., work, parenting, other) related activity (e.g., standing 4-6 hours everyday, walking 1-2 miles everyday),
Exercise related activity,
   Eating
      Restrictive behaviors (e.g., dieting, fasting, quantity restricting, content restricting),
      Excessive behaviors (e.g., eating until feeling sick)
      Purging behaviors (e.g., vomiting, taking laxatives)
      Conditions of eating (e.g., eating alone, eating at night),
   Self-care
Social & connectional health, e.g., relationship status
Occupational health, such as
   Education level,
   Employment status,
Household health, such as
   Household income,
   Household debt,
   Food security,
   Housing security,
   Future security,
Financial health, such as
   Household income,
   Household financial distress,
Genetics and inherited health, such as
   Parents' health history for biological (if applicable) and/or for adoptive parents,
   Siblings' health history for biological (if applicable) and/or adoptive (if applicable), and/or siblings raised in same household (if applicable),
Societal & structural health, such as
   Race/Ethnicity,
   Zip Code,
   Immigration status,
   English proficiency,
Partner health, such as
   Partner's behavioral health history,
   Partner's physical health history,
Children's health: Each child's health history (e.g., biological, adopted, fostered, step, under guardianship)
   Each child's behavioral health history,
   Each child's physical health history.

Example raw data received from the individual or a member within the individual's circle of friends and families include on-demand (i.e., unprompted) event diaries, voice data, audio data, the individual/circle members' interactions with the platform such as questionnaires filled by the user such as quantitative and qualitative data on individual's relationships within different social structures, questionnaires filled by one or more members of the individual's circle of friends and family, narrative entries on relationships, voice data on narrative user entries on relationships, etc.

Example pre-processing of event diaries include extracting the following information from unstructured text: word-choice, number of words used, repeated words, completion duration, keyword analysis, time of entry, etc., and further processing of this data for sentiment analysis, emotion detection, emergency risk states, etc. by the use of ML models. Example pre-processing of voice data includes extracting the following information from a voice recording: the word-choice, number of word uses, pitch analysis, pace of talk, intonation etc., and further processing of this data for sentiment analysis, emotion detection, emergency risk states, etc. by the use of ML models.

The circle of friends and family can include people having i) immediate family relations, ii) inner circle relations, iii) outer circle relations, and/or iv) community relations with the individual.

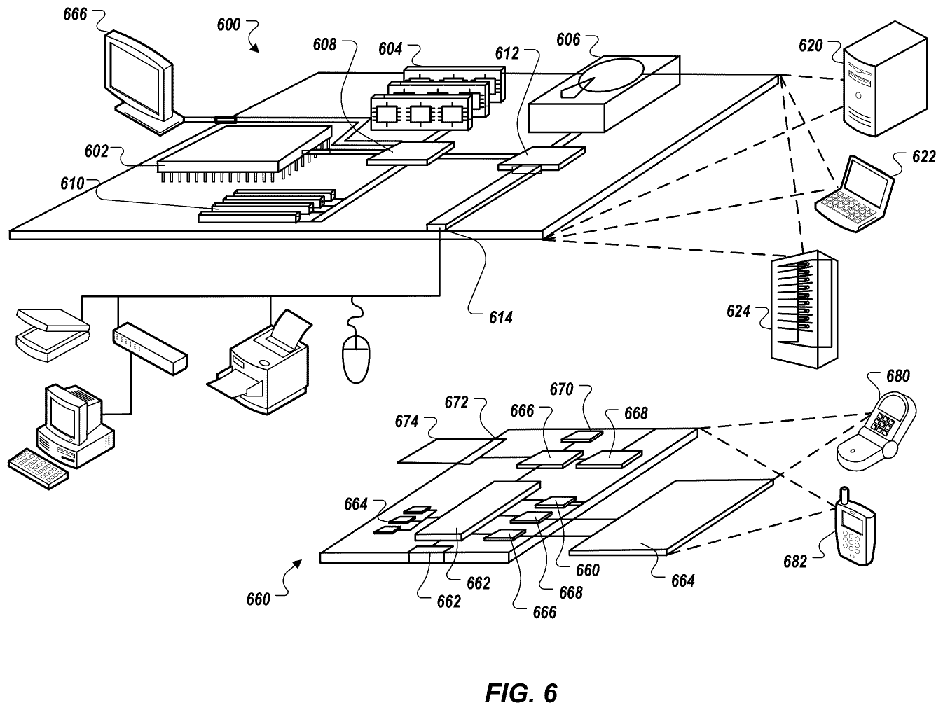

FIG. 6 shows an example of a computing device 600 and an example of a mobile computing device that can be used to implement the techniques described here. For example, the system 122 depicted in FIG. 1 can be in the form of the computing device 600, the mobile computing device 660, or a combination of them. User device 104 depicted in FIG. 1 can be in the form of the computing device 600, or the mobile device 660. The computing device 600 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 600 includes a processor 602, a memory 604, a storage device 606, a high-speed interface 608 connecting to the memory 604 and multiple high-speed expansion ports 610, and a low-speed interface 612 connecting to a low-speed expansion port 614 and the storage device 606. Each of the processor 602, the memory 605, the storage device 606, the high-speed interface 608, the high-speed expansion ports 610, and the low-speed interface 612, are interconnected using various busses, and can be mounted on a common motherboard or in other manners as appropriate. The processor 602 can process instructions for execution within the computing device 600, including instructions stored in the memory 604 or on the storage device 606 to display graphical information for a GUI on an external input/output device, such as a display 616 coupled to the high-speed interface 608. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 604 stores information within the computing device 600. In some implementations, the memory 604 is a volatile memory unit or units. In some implementations, the memory 604 is a non-volatile memory unit or units. The memory 604 can also be another form of computer-readable medium, such as a magnetic or optical disk.

The storage device 606 is capable of providing mass storage for the computing device 600. In some implementations, the storage device 606 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 605, the storage device 606, or memory on the processor 602.

The high-speed interface 608 manages bandwidth-intensive operations for the computing device 600, while the low-speed interface 612 manages lower bandwidth-intensive operations. Such allocation of functions is exemplary only. In some implementations, the high-speed interface 608 is coupled to the memory 605, the display 616 (e.g., through a graphics processor or accelerator), and to the high-speed expansion ports 610, which can accept various expansion cards (not shown). In the implementation, the low-speed interface 612 is coupled to the storage device 606 and the low-speed expansion port 615. The low-speed expansion port 615, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 600 can be implemented in a number of different forms, as shown in the figure. For example, it can be implemented as a standard server 620, or multiple times in a group of such servers. In addition, it can be implemented in a personal computer such as a laptop computer 622. It can also be implemented as part of a rack server system 625. Alternatively, components from the computing device 600 can be combined with other components in a mobile device (not shown), such as a mobile computing device 660. Each of such devices can contain one or more of the computing device 600 and the mobile computing device 660, and an entire system can be made up of multiple computing devices communicating with each other.

The mobile computing device 660 includes a processor 662, a memory 665, an input/output device such as a display 655, a communication interface 666, and a transceiver 668, among other components. The mobile computing device 660 can also be provided with a storage device, such as a micro-drive or other device, to provide additional storage. Each of the processor 662, the memory 665, the display 655, the communication interface 666, and the transceiver 668, are interconnected using various buses, and several of the components can be mounted on a common motherboard or in other manners as appropriate.

The processor 662 can execute instructions within the mobile computing device 660, including instructions stored in the memory 665. The processor 662 can be implemented as a chipset of chips that include separate and multiple analog and digital processors. The processor 662 can provide, for example, for coordination of the other components of the mobile computing device 660, such as control of patient interfaces, applications run by the mobile computing device 660, and wireless communication by the mobile computing device 660.

The processor 662 can communicate with a patient through a control interface 668 and a display interface 666 coupled to the display 655. The display 664 can be, for example, a TFT (Thin-Film-Transistor Liquid Crystal Display) display or an OLED (Organic Light Emitting Diode) display, or other appropriate display technology. The display interface 666 can comprise appropriate circuitry for driving the display 664 to present graphical and other information to a patient. The control interface 668 can receive commands from a patient and convert them for submission to the processor 662. In addition, an external interface 662 can provide communication with the processor 662, so as to enable near area communication of the mobile computing device 660 with other devices. The external interface 662 can provide, for example, for wired communication in some implementations, or for wireless communication in other implementations, and multiple interfaces can also be used.

The memory 664 stores information within the mobile computing device 660. The memory 664 can be implemented as one or more of a computer-readable medium or media, a volatile memory unit or units, or a non-volatile memory unit or units. An expansion memory 674 can also be provided and connected to the mobile computing device 660 through an expansion interface 672, which can include, for example, a SIMM (Single In Line Memory Module) card interface. The expansion memory 674 can provide extra storage space for the mobile computing device 660, or can also store applications or other information for the mobile computing device 660. Specifically, the expansion memory 674 can include instructions to carry out or supplement the processes described above, and can include secure information also. Thus, for example, the expansion memory 674 can be provided as a security module for the mobile computing device 660, and can be programmed with instructions that permit secure use of the mobile computing device 660. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 665, the expansion memory 675, or memory on the processor 662. In some implementations, the computer program product can be received in a propagated signal, for example, over the transceiver 668 or the external interface 662.

The mobile computing device 660 can communicate wirelessly through the communication interface 666, which can include digital signal processing circuitry where necessary. The communication interface 666 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 668 using a radio-frequency. In addition, short-range communication can occur, such as using a Bluetooth, WiFi, or other such transceiver (not shown). In addition, a GPS (Global Positioning System) receiver module 670 can provide additional navigation- and location-related wireless data to the mobile computing device 660, which can be used as appropriate by applications running on the mobile computing device 660.

The mobile computing device 660 can also communicate audibly using an audio codec 660, which can receive spoken information from a patient and convert it to usable digital information. The audio codec 660 can likewise generate audible sound for a patient, such as through a speaker, e.g., in a handset of the mobile computing device 660. Such sound can include sound from voice telephone calls, can include recorded sound (e.g., voice messages, music files, etc.) and can also include sound generated by applications operating on the mobile computing device 660.

The mobile computing device 660 can be implemented in a number of different forms, as shown in the figure. For example, it can be implemented as a cellular telephone 680. It can also be implemented as part of a smart-phone 682, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a patient (or a user, in general), the systems and techniques described here can be implemented on a computer having a display device (e.g., a CRT (cathode ray tube) or LCD (liquid crystal display) monitor) for displaying information to the patient and a keyboard and a pointing device (e.g., a mouse or a trackball) by which the patient can provide input to the computer. Other kinds of devices can be used to provide for interaction with a patient as well; for example, feedback provided to the patient can be any form of sensory feedback (e.g., visual feedback, auditory feedback, or tactile feedback); and input from the patient can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical patient interface or a Web browser through which a patient can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

A number of implementations of the present disclosure have been described. Nevertheless, it will be understood that various modifications may be made without departing from the spirit and scope of the present disclosure. Accordingly, other implementations are within the scope of the following claims.

Some implementations include a computer-implemented method executable by a computing system. The method includes receiving, from a plurality of devices, respective pieces of input data regarding a first status of a target individual, each piece of input data is received from a respective device in the plurality of devices that is remote from the computing system. Multiple devices in the plurality of devices are associated with respective people in a circle of individuals that each has a respective specified relationship with the target individual. At least a piece of input data is received in response to an interaction of a person with a respective device in the plurality of devices, the person being in the circle of individuals.

The method further includes pre-processing the pieces of input data to obtain input for a machine learning model. The pre-processing includes extracting from a piece of input data multiple features associated with a particular input module in one or more input modules of the machine learning model, each input module being associated with a respective marker, and transforming each extracted feature to a respective input feature that has a respective format associated with the input module, wherein at least two input modules are associated with different formats, and wherein at least one extracted feature is transformed to the different formats associated with the at least two input modules.

The method further includes inputting the pre-processed pieces of input to respective input modules of the machine learning model, wherein each input module has an input layer including the input features obtained through the pre-processing, wherein an input layer of a first module is different from an input layer of a second module; receiving, from the machine learning model, an output prediction for the target individual; analyzing the output to determine an urgency associated with the prediction, wherein the urgency is determined in response to determining that a probability of the prediction is more than a pre-specified threshold value; and generating and sending an alert to at least one other individual, notifying them about the urgency, wherein the alert includes an action recommendation.

The output prediction of the machine learning model can include respective probabilities that multiple events happen to the target individual. In some implementations, an input module in the one or more input modules provides outputs including respective values that each indicates a probability of a respective event in the multiple events for the target individual. In some implementations, each of the input modules provides respective outputs for the same events as the multiple events for which the machine learning model provides the output.

The interaction of the person with the respective device can include entering information on an application running on the respective device.

In some implementations, at least a few devices in the multiple devices are respective sensors measuring or monitoring respective parameters of the target individual.

In some implementations, at least two pieces of input data have different formats, and wherein the formats include one or more of voice, text, selection of an item on a respective device in the multiple devices.

The presented computing system can include one or more computers, and one or more computer-readable storage devices storing instructions that are operable, when executed by the one or more computers, to cause the one or more computers to perform operations in accordance with implementations of the methods provided herein.

The present disclosure also provides one or more non-transitory computer-readable storage medium coupled to one or more processors and having instructions stored thereon which, when executed by the one or more processors, cause the one or more processors to perform operations in accordance with implementations of the methods provided herein.

What is claimed is:

1. A computer implemented method executed on a computing system, the method comprising:
receiving, from a plurality of devices, respective pieces of input data regarding a first status of a target individual, each piece of input data is received from a respective device in the plurality of devices that is remote from the computing system,
wherein multiple devices in the plurality of devices are associated with respective people in a circle of individuals that each has a respective specified relationship with the target individual, and
wherein at least a piece of input data is received in response to an interaction of a person with a respective device in the plurality of devices, the person being in the circle of individuals;
pre-processing the pieces of input data to obtain input for a machine learning model, wherein the pre-processing includes:
extracting from a piece of input data multiple features associated with a particular input module in one or more input modules of the machine learning model, each input module being associated with a respective marker, and
transforming each extracted feature to a respective input feature that has a respective format associated with the input module, wherein at least two input modules are associated with different formats, and wherein at least one extracted feature is transformed to the different formats associated with the at least two input modules;
inputting the pre-processed pieces of input to respective input modules of the machine learning model, wherein each input module has an input layer including the input features obtained through the pre-processing, wherein an input layer of a first module is different from an input layer of a second module;
receiving, from the machine learning model, an output prediction for the target individual;
analyzing the output to determine an urgency associated with the prediction, wherein the urgency is determined in response to determining that a probability of the prediction is more than a pre-specified threshold value; and
generating and sending an alert to at least one other individual, notifying them about the urgency, wherein the alert includes an action recommendation.

2. The method of claim 1, wherein the output prediction of the machine learning model includes respective probabilities that multiple events happen to the target individual.

3. The method of claim 2, wherein an input module in the one or more input modules provides outputs including respective values that each indicates a probability of a respective event in the multiple events for the target individual.

4. The method of claim 2, wherein each of the input modules provides respective outputs for the same events as the multiple events for which the machine learning model provides the output.

5. The method of claim 1, wherein the interaction of the person with the respective device includes entering information on an application running on the respective device.

6. The method of claim 1, wherein at least a few devices in the multiple devices are respective sensors measuring or monitoring respective parameters of the target individual.

7. The method of claim 1, wherein at least two pieces of input data have different formats, and wherein the formats include one or more of voice, text, selection of an item on a respective device in the multiple devices.

8. A computing system comprising:
one or more processors; and
one or more computer memories connected to communicate with the one or more processors and storing instructions, that when executed by the one or more processors, cause the system to:
receiving, from a plurality of devices, respective pieces of input data regarding a first status of a target individual, each piece of input data is received from a respective device in the plurality of devices that is remote from the computing system,
wherein multiple devices in the plurality of devices are associated with respective people in a circle of individuals that each has a respective specified relationship with the target individual, and
wherein at least a piece of input data is received in response to an interaction of a person with a respective device in the plurality of devices, the person being in the circle of individuals;
pre-processing the pieces of input data to obtain input for a machine learning model, wherein the pre-processing includes:
extracting from a piece of input data multiple features associated with a particular input module in one or more input modules of the machine learning model, each input module being associated with a respective marker, and
transforming each extracted feature to a respective input feature that has a respective format associated with the input module, wherein at least two input modules are associated with different formats, and wherein at least one extracted feature is transformed to the different formats associated with the at least two input modules;
inputting the pre-processed pieces of input to respective input modules of the machine learning model, wherein each input module has an input layer including the input features obtained through the pre-processing, wherein an input layer of a first module is different from an input layer of a second module;
receiving, from the machine learning model, an output prediction for the target individual;
analyzing the output to determine an urgency associated with the prediction, wherein the urgency is determined in response to determining that a probability of the prediction is more than a pre-specified threshold value; and
generating and sending an alert to at least one other individual, notifying them about the urgency, wherein the alert includes an action recommendation.

9. The system of claim 8, wherein the output prediction of the machine learning model includes respective probabilities that multiple events happen to the target individual.

10. The system of claim 9, wherein an input module in the one or more input modules provides outputs including respective values that each indicates a probability of a respective event in the multiple events for the target individual.

11. The system of claim 9, wherein each of the input modules provides respective outputs for the same events as the multiple events for which the machine learning model provides the output.

12. The system of claim 8, wherein the interaction of the person with the respective device includes entering information on an application running on the respective device.

13. The system of claim 8, wherein at least a few devices in the multiple devices are respective sensors measuring or monitoring respective parameters of the target individual.

14. The system of claim 8, wherein at least two pieces of input data have different formats, and wherein the formats include one or more of voice, text, selection of an item on a respective device in the multiple devices.

15. A non-transitory, computer-readable medium storing one or more instructions executable by a computer system to perform operations comprising:
receiving, from a plurality of devices, respective pieces of input data regarding a first status of a target individual, each piece of input data is received from a respective device in the plurality of devices that is remote from the computing system,
wherein multiple devices in the plurality of devices are associated with respective people in a circle of individuals that each has a respective specified relationship with the target individual, and
wherein at least a piece of input data is received in response to an interaction of a person with a respective device in the plurality of devices, the person being in the circle of individuals;
pre-processing the pieces of input data to obtain input for a machine learning model, wherein the pre-processing includes:
extracting from a piece of input data multiple features associated with a particular input module in one or more input modules of the machine learning model, each input module being associated with a respective marker, and
transforming each extracted feature to a respective input feature that has a respective format associated with the input module, wherein at least two input modules are associated with different formats, and wherein at least one extracted feature is transformed to the different formats associated with the at least two input modules;
inputting the pre-processed pieces of input to respective input modules of the machine learning model, wherein each input module has an input layer including the input features obtained through the pre-processing, wherein an input layer of a first module is different from an input layer of a second module;
receiving, from the machine learning model, an output prediction for the target individual;
analyzing the output to determine an urgency associated with the prediction, wherein the urgency is determined in response to determining that a probability of the prediction is more than a pre-specified threshold value; and
generating and sending an alert to at least one other individual, notifying them about the urgency, wherein the alert includes an action recommendation.

16. The non-transitory, computer-readable medium of claim 15, wherein the output prediction of the machine learning model includes respective probabilities that multiple events happen to the target individual.

17. The non-transitory, computer-readable medium of claim 16, wherein an input module in the one or more input modules provides outputs including respective values that each indicates a probability of a respective event in the multiple events for the target individual.

18. The non-transitory, computer-readable medium of claim 16, wherein each of the input modules provides respective outputs for the same events as the multiple events for which the machine learning model provides the output.

19. The non-transitory, computer-readable medium of claim 15, wherein the interaction of the person with the respective device includes entering information on an application running on the respective device.

20. The non-transitory, computer-readable medium of claim 15, wherein at least a few devices in the multiple devices are respective sensors measuring or monitoring respective parameters of the target individual.
